(19) United States
(12) Patent Application Publication
(10) Pub. No.: US 2013/0190772 A1
(43) Pub. Date: Jul. 25, 2013

(54) Elastic Guide Wire for Spinal Surgery

(71) Applicant: MIS SURGICAL, LLC, Scottsdale, AZ (US)

(72) Inventor: Todd Doerr, Scottsdale, AZ (US)

(73) Assignee: MIS SURGICAL, LLC, Scottsdale, AZ (US)

(21) Appl. No.: 13/748,676

(22) Filed: Jan. 24, 2013

Related U.S. Application Data
(60) Provisional application No. 61/590,112, filed on Jan. 24, 2012.

(51) Int. Cl. A61B 17/86; A61B 17/56

(52) U.S. Cl. CPC A61B 17/86; A61B 17/56
USPC 606/104

(57) ABSTRACT

An elastic alloy wire is used to guide pedicle screw insertion during spinal surgery of a patient. Once the pedicle screw is affixed to the vertebra, the elastic alloy wire is bent away from the intraoperative region, providing the surgeon and surgeon's assistant better access to the intraoperative region and the anatomy of the patient.

### CROSS-REFERENCE TO RELATED APPLICATIONS

This application claims priority to, and the benefit of, U.S. Patent Application Ser. No. 61/590,112, entitled “Elastic Guide Wire for Spinal Surgery,” filed Jan. 24, 2012, the entire contents of which are incorporated herein by reference.

### FIELD

Embodiments generally relate to surgical methods, systems, assemblies, and devices, and more particularly, to spinal surgery methods, systems, assemblies, and devices; and most particularly to use of an elastic alloy wire for spinal surgery.

### BACKGROUND

Patients that suffer from a degenerative or deformative spinal condition, such as a herniated nucleus pulposus or scoliosis, often undergo spinal stabilization and/or spinal fusion to correct the condition. Spinal stabilization is a surgical technique that stabilizes spinal vertebrae without fusing the vertebrae together. Spinal fusion (“spondylodesis” or “spondylosyndesis”) on the other hand, entails permanently affixing two or more vertebrae together (“arthrodesis”). Both procedures use pedicle screws that are used to anchor the vertebrae.

The success in such orthopedic surgical procedures relies on accuracy. For example, malpositioning of a pedicle screw during spinal surgery can cause perforation of the cortex and impingement on adjacent structures that result in neurological or vascular injury to the patient (e.g., human or animal). Consequently, surgeons use fluoroscopy and radiography, along with surgical tools such as guide wires, to guide placement and positioning of the pedicle screws. When multiple guide wires are used with pedicle screws, the operating region becomes congested and surgery becomes cumbersome.

It would, therefore, be desirable to have methods, systems, assemblies, and devices for use in spinal surgical procedures that address the above issues.

### SUMMARY

In certain embodiments, a method for spinal surgery involving a plurality of pedicle screw insertions is disclosed. For each of the plurality of pedicle insertion sites, a proximal end of an elastic alloy wire is temporarily inserted into a respective pedicle of a vertebra. The elastic alloy wire does not kink when bent. A distal end of the elastic alloy wire is inserted into a lumen of a respective pedicle screw that is moved along the elastic alloy wire toward the proximal end of the elastic alloy wire. The elastic alloy wire removed from the pedicle of the vertebra.

In certain embodiments, a method for spinal surgery involving inserting a plurality of pedicle screws into a plurality of respective pedicles is disclosed. For each of the plurality of pedicle screws a proximal end of a titanium-nickel alloy wire is temporarily inserted into a respective pedicle of a vertebra. The proximal end of the titanium-nickel alloy wire includes threading and the titanium-nickel alloy wire does not kink when bent. The titanium-nickel alloy wire is twisted along its long axis to push at least a portion of the threading into the pedicle. A distal end of titanium-nickel alloy wire is inserted into a lumen of a respective pedicle screw and the respective pedicle screw is moved along the titanium-nickel alloy wire toward the proximal end. The titanium-nickel alloy wire is untwisted free from the pedicle of the vertebra.

In certain embodiments, a wire to guide an entry site of a pedicle screw used in spinal surgery is disclosed. The wire comprises an elastic alloy having an elasticity that enables a distal end of the wire to be bent away from an intraoperative region during the spinal surgery without forming a kink along a length of the wire. A proximal end of the wire has threading. The wire is dimensioned to: fit into a lumen of a pedicle screw; and allow the pedicle screw to glide along the length of the wire from a distal end toward the proximal end of the wire.

### BRIEF DESCRIPTION OF THE DRAWINGS

The invention will be better understood from a reading of the following detailed description taken in conjunction with the drawings in which like reference designators are used to designate like elements, and in which:

FIG. 1A is a side view of a human vertebral column;

FIG. 1B is a top view of a vertebra;

FIGS. 2A-2C are each a schematic illustrating a spinal stabilization system;

FIG. 2D is a schematic illustrating a spinal fusion fixation system;

FIG. 3A is a side view of an exemplary pedicle screw;

FIG. 3B is a top view of a vertebra including a pair of substantially, permanently affixed pedicle screws;

FIG. 4A is an illustration of an elastic alloy wire;

FIG. 4B is an illustration of an expanded view of a proximal end of the elastic alloy wire of FIG. 4A;

FIG. 4C is an illustration of an elastic alloy wire inserted into a pedicle screw;

FIGS. 5A and 5B are each a schematic illustrating an exemplary spinal fusion fixation system including a plurality of elastic alloy wires that are bent away from the intraoperative region;

FIG. 6 is stress/strain graph for Applicant's titanium-nickel alloy; and

FIG. 7 illustrates a flow chart of an exemplary method for spinal surgery involving the use of one or more elastic alloy wires.

### DETAILED DESCRIPTION

The invention is described in preferred embodiments in the following description with reference to the FIGs., in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” “in certain embodiments,” and similar language throughout this specification may, but do not necessarily, all refer to the same embodiment. It is noted that, as used in this description, the singular forms “a,” “an” and “the” include plural referents unless the context clearly dictates otherwise.

The described features, structures, or characteristics of the invention may be combined in any suitable manner in one or more embodiments. In the following description, numerous specific details are recited to provide an understanding of various embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

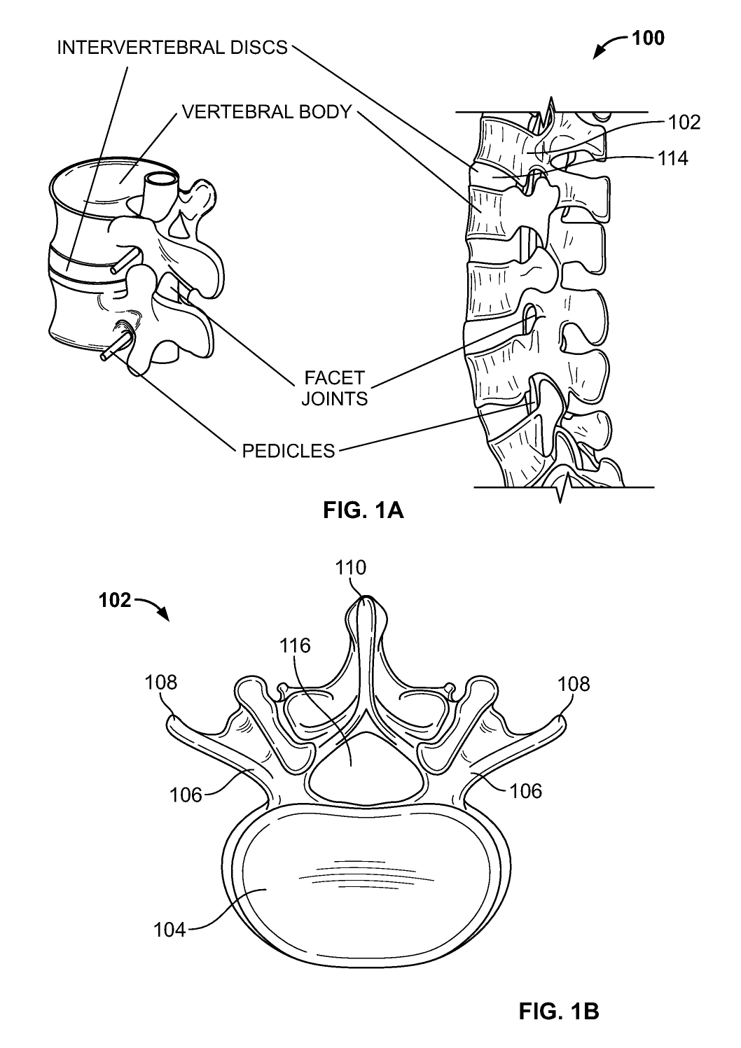

Referring to FIGS. 1A and 1B, a vertebral column 100 of a human patient includes twenty four articulating vertebrae 102 and nine fused vertebrae in the sacrum and coccyx (not shown). The anterior of the vertebrae 102 includes a vertebral body 104 comprising a pair of pedicles 106. The pedicles 106 are strong, cylindrical, anatomic bridges between the dorsal spinal elements 108 and 110 and the vertebral body and consist of a strong shell of cortical bone and a core of cancellous bone. The transverse pedicle width is narrower than the sagittal pedicle width (height) except in the lower lumbar spine. Most of the pedicles 106 below the thoracic are greater than 7 mm in transverse diameter. In addition, the transverse pedicle 106 width increases from lumbar vertebrae to the sacral segments of the vertebral column 100.

The vertebra 102 also has two transverse processes 108 and a spinous process 110 at the posterior side. An intervertebral disc 114 lies between adjacent vertebrae in the vertebral column 100. The vertebral column 100 surrounds and protects the spinal cord. Nerves (not shown) branch out from the spinal cord and exit the vertebral column 100 between each vertebra through a hole called the vertebral foramen 116.

Abnormalities in the vertebral column 100 that are congenital (e.g., scoliosis) or due to disease (e.g., herniated disc), trauma (e.g., whiplash), or aging (e.g., arthritis) may require corrective measures through surgical intervention. In some instances, the surgical intervention includes substantially permanently affixing a surgical object, such as a pedicle screw, rod, lateral connector, or an implant, into a bone of the patient.

Spinal stabilization and spinal fusion are surgical techniques that create a union between adjacent vertebrae 102 as a means to correct dysfunction or instability of the vertebral column 100. These techniques provide immobilization and stabilization of spinal segments in the treatment of the acute and chronic instabilities or deformities of the thoracic, lumbar, and sacral spine, such as: fracture, dislocation, degenerative spondylolisthesis with evidence of neurologic impairment, scoliosis, kyphosis, spinal tumor, and failed previous fusion (pseudarthrosis). For each technique, the surgeon builds a spinal implant system to fit the patient's anatomical and physiological requirements. For example, depending on the patient's physiological needs, the spinal implant system comprises a combination of anchors (e.g., bolts, hooks, and/or pedicle screws); interconnection mechanisms incorporating nuts, screws, sleeves, or bolts; longitudinal members (e.g., plates, rods, and/or plate/rod combinations); and/or transverse connectors.

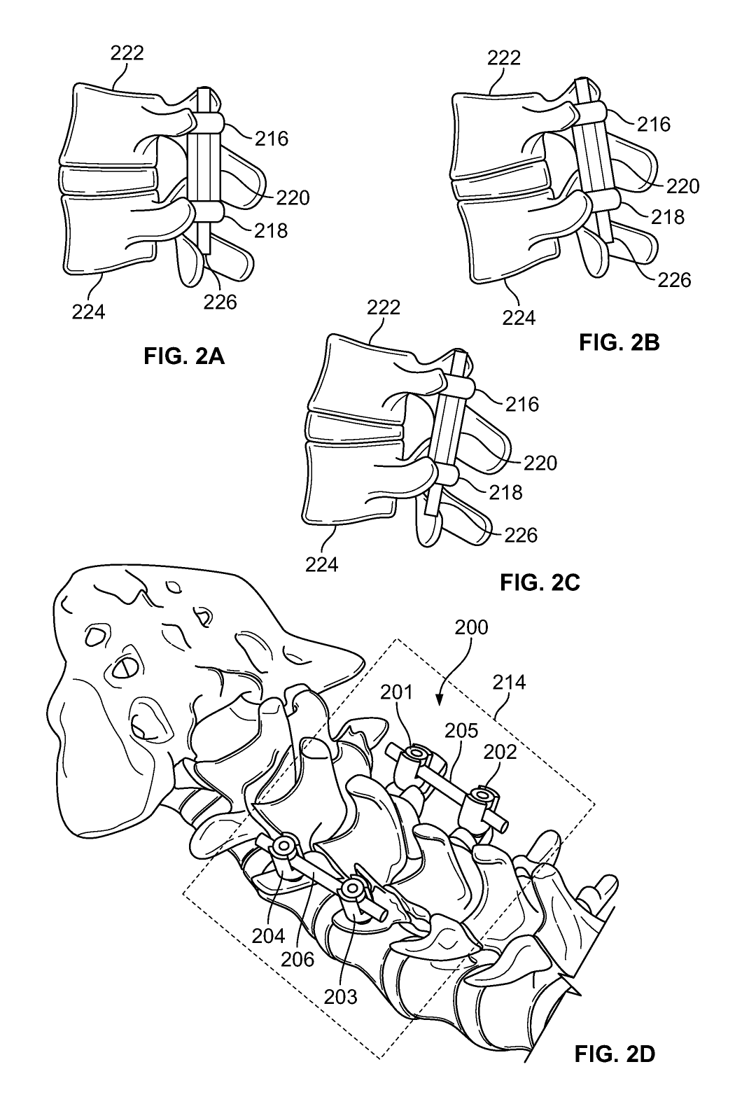

Referring to FIGS. 2A-2C, a spinal stabilization technique uses an exemplary spinal implant system including pedicle screws 216 and 218; a spacer 220 made of surgical polyurethane; and a cord 226 made of nylon, for example. The pedicle screws 216 and 218 anchor into the pedicles 106 of respective vertebrae 222 and 224. The spacer 220 flexibly holds two vertebrae 222 and 224 together in various anatomical positions relative to one another, such as in a neutral position of FIG. 2A, flexed position of FIG. 2B, and an extended position in FIG. 2C. The cord 226 runs through the spacer 220 and is taut to limit flexion, extension, or rotation of the two vertebrae 222 and 224.

FIG. 2D illustrates an intraoperative region 214 for a patient undergoing spinal fusion surgery using pedicle screws. Typically, the soft tissue of the patient (not shown) is retracted to expose the spine of the patient. Here, spinal implant system 200 is affixed to the boney structure of the patient. The spinal implant system 200 includes pedicle screws 201, 202, 203, and 204; and rods 205 (pedicle screws on patient's left side) and 206 (pedicle screws on patient's right side). The pedicle screws 202 & 203 are placed into a first vertebra and pedicle screws 201 and 204 are placed in a second, more caudal vertebra, both of which are then fused to one another, adding extra support and strength to the fusion. The rod 205 connects the pedicle screws 201 and 202 together and rod 206 connects pedicle screws 203 and 204 together. Here, the spinal implant system 200 prevents movement of the first and second vertebrae relative to one another and allows the bone graft to heal, for example. In some implementations, spinal implant system 200 includes one or more lateral connectors (not shown) that interconnect two or more rods on the patient's side (e.g., multiple rods 205 on patient's left side or multiple rods 206 on patient's right side).

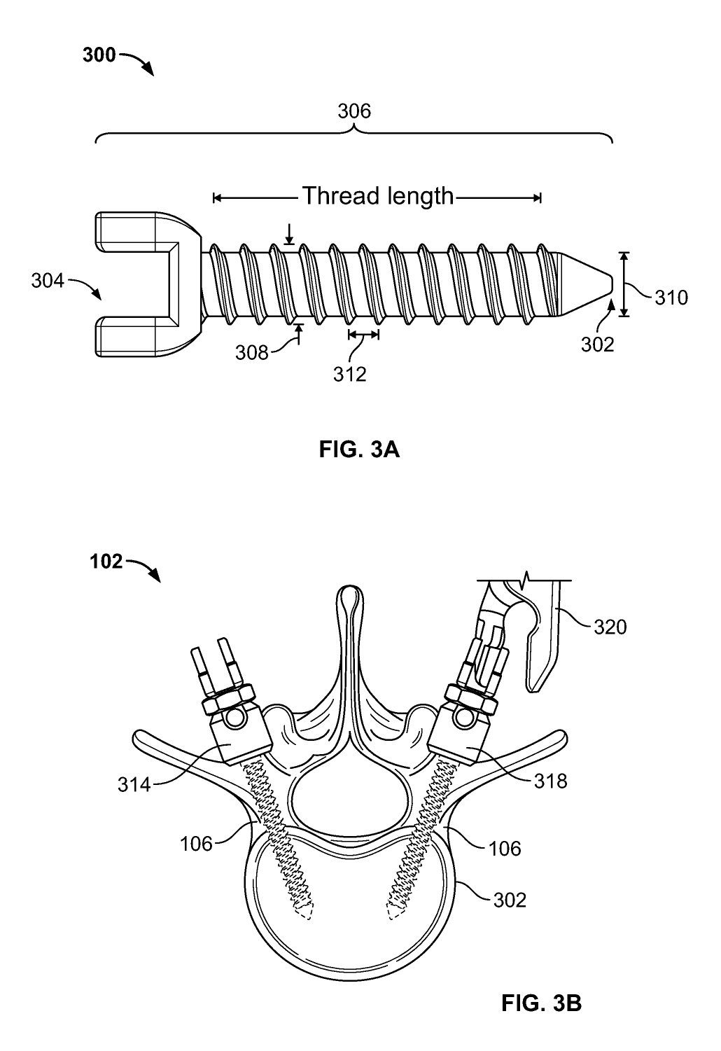

Both spinal stabilization and spinal fusion techniques utilize pedicle screws. Referring to FIG. 3A illustrates a pedicle screw 300. The pedicle screw 300 is made from a variety of materials, including alloys such as 22Cr-13Ni-5Mn stainless steel, 316L stainless steel, 316LVM stainless steel, and Ti-6Al-4V; and unalloyed titanium. The length 306 of the pedicle screw 300 depends on its application. In certain embodiments, the pedicle screw 300 length ranges from about 30 mm to about 60 mm with a major diameter 308 range from about 4.0 mm to about 8.5 mm. In the illustrated examples of FIGS. 3A and 3B, a proximal end 302 of the pedicle screw 300 is conical in shape and its distal end 304 is adapted to couple with turning tool 320. The body of the pedicle screw 300 is threaded, having a minor 310 and a major 308 diameter. The pitch 312 is progressive or V-Shaped, for example. The interior of the pedicle screw has a lumen 322 that extends from the distal 304 to the proximal 302 end of the pedicle screw 300 with corresponding openings.

Referring to FIG. 3B, two pedicle screws 300, shown as pedicle screws 314 and 318, are affixed into a vertebra 102, respectively. Although a single size and angulation of pedicle screws 314 and 318 are illustrated in FIG. 3B, the pedicle screw size and insertion angulation can vary, depending on the region of the spinal column that is operated upon. This is because the width of the pedicle 106 increases in the lower lumbar spine and is variable in the thoracic spine. Typically, the pedicle width is more important than pedicle height for pedicle screw placement.

The pedicles 106 of the vertebra are the strongest points of attachment of the spine allowing for a significant amount of force to be applied when inserting the pedicle screws 314 and 318 into the spine without failure of the bone-metal junction. In FIG. 3B, the pedicle screws 314 and 318 are shown as traversing the vertebra rigidly, stabilizing both the ventral and dorsal aspects of the vertebra.

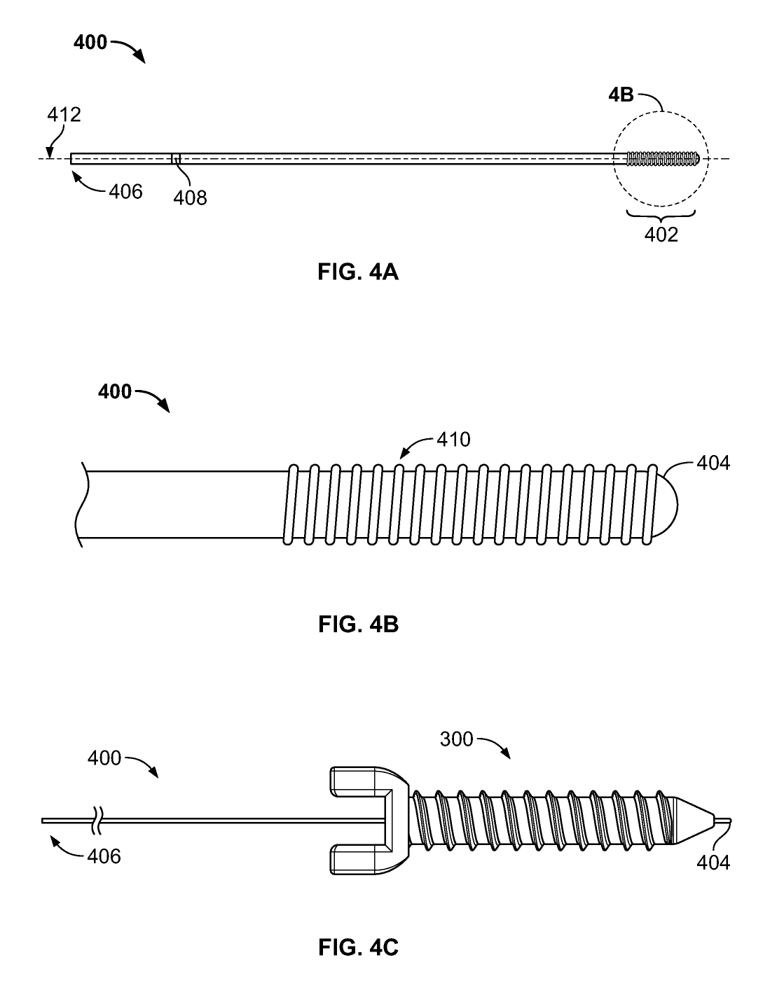

In certain embodiments, a wire, such as a Kirschner wire or K-wire, is utilized during spinal surgery to position, and guide an entry site for, a pedicle screw. Referring to FIGS. 4A and 4B, wire 400 comprises a proximal 402 and a distal 406 end (not shown to scale). The wire 400 of FIG. 4B further comprises threading 410 with a corresponding pitch, which in certain embodiments, extends to about the tip 404 of the wire 400. Other orientation of the threading 410 and tip 404 is also contemplated, such as a diameter of the threading 410 being larger or smaller than that of the tip 404. The threading stabilizes the wire 400 into the bone and prevents the wire 400 from backing-out of bone during surgery.

In FIG. 4C, the distal end 406 of the wire 400 is inserted through the lumen of the pedicle screw 300 and the pedicle screw is moved along the length of the wire towards the proximal end 402. For illustrative purposes, the tip 404 is shown as protruding past the proximal end 302 of the pedicle screw 300.

In certain embodiments, wire 400 includes a visible position indicating mark 408 at the distal end 406 of the wire 400, such as at about 50 mm from the distal edge of the wire 400 and/or about 450 mm from the tip 404 at the proximal end 402. Position indicating mark 408 is used to determine the position and/or orientation of the wire 400 during surgery. To illustrate, an imaging device detects the position and/or orientation of the position indicating mark 408 relative to a known coordinate system within an intraoperative region. The position indicating mark 408 along with other image reference points or bony markers are used to determine a location of the proximal end of the wire placed into the pedicle, a trajectory along the wire, and/or spacing between a plurality of wires placed into their respective pedicles.

The wire 400 has a shape and dimension to match the dimensions of the corresponding pedicle screw used in the spinal implant system and the desired level of control of the corresponding pedicle screws during spinal surgery. For example, the wire 400 is cylindrical in shape having a length of about 400 mm to about 700 mm, such as about 500 mm, and a diameter in a range of about 1.0 mm to about 5.0 mm, such as about 1.0 mm to about 3.0 mm, or about 1.2 mm to about 2.6 mm. Other shapes and dimensions are also contemplated, for example in certain embodiments, the wire 400 has an oval, elliptical, square, pentagon, and/or hexagon cross section. In certain embodiments, the wire has a tip at the proximal end that is rounded, blunt, sharp, beveled, and a combination thereof.

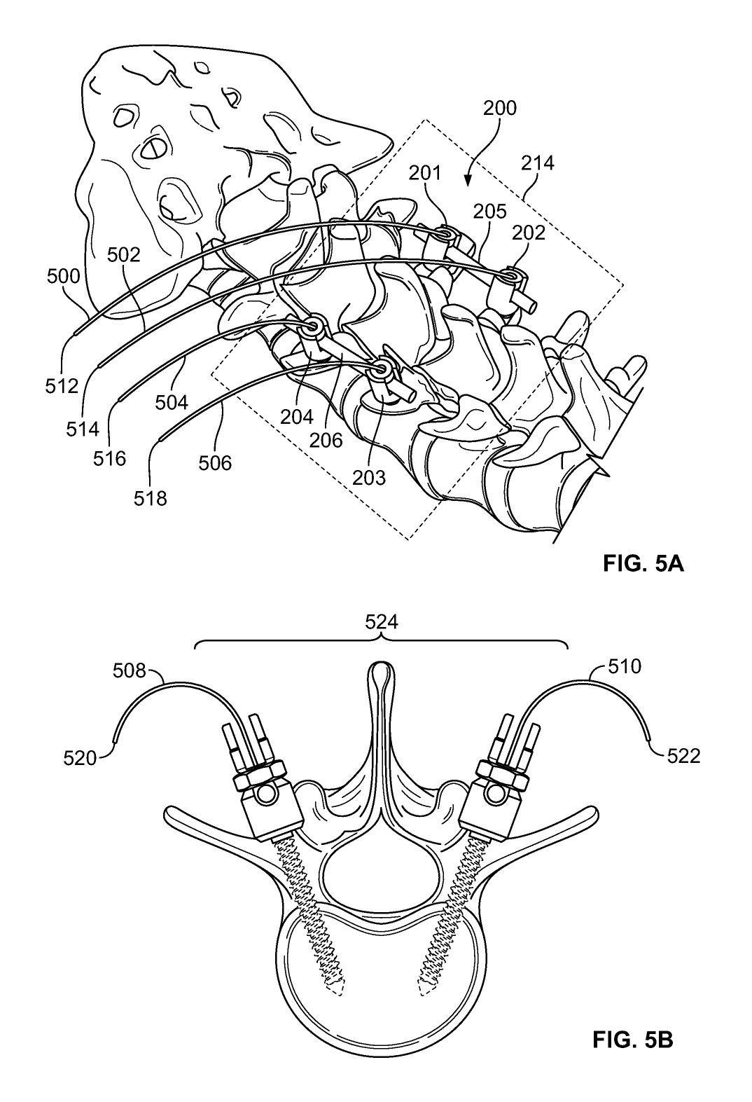

In certain embodiments, the wire 400 is made out of material that is elastic, such as an elastic alloy, that it does not kink when bent (“kink resistant”) wire. This elasticity allows the wire to be bent away from the intraoperative region 214 during surgery, giving the surgeon and/or surgeon's assistants better access to the intraoperative region. When a bending force exerted on the wire 400 is released, the wire 400 returns to about its original position prior to bending. To illustrate, in FIG. 5A, a plurality of wires, shown as elastic alloy wires 500-506, are each bent away from the intraoperative region 214 of FIG. 2D. In the illustrated embodiment of FIG. 5A, the distal ends of the wires 500-506 (e.g., distal end 512-518, respectively) are shown as being disposed downwardly towards the patient and away from the intraoperative region 214. Similarly in FIG. 5B (not drawn to scale), the distal ends 520 and 522 of elastic alloy wires 508 and 510, respectively, are shown as bending downwardly and away from the intraoperative region 524.

In certain embodiments, the elastic alloy wires (e.g., wires 400 or 500-512) are each formed from a kink resistant titanium-nickel alloy comprising about equal atomic percentages of titanium and nickel. For illustrative purposes only, in certain embodiments, the elastic alloy wire (e.g., wires 400 or 500-512) comprises about the following:

| Element | Weight % |
|---|---|
| Nickel | 54 to 57 (Reference) |
| Carbon | <.05 (500 ppm maximum) |
| Cobalt | <.05 (500 ppm maximum) |
| Copper | <.01 (100 ppm maximum) |
| Chromium | <.01 (100 ppm maximum) |
| Hydrogen | <.005 (50 ppm maximum) |
| Iron | <.05 (500 ppm maximum) |
| Niobium | <.025 (250 ppm maximum) |
| Nitrogen plus Oxygen | <.05 (500 ppm maximum) |
| Any Single Trace Element | <.1 |
| Total Trace Element | <.25 |
| Titanium | Balance |

To illustrate, the elastic alloy wire (e.g., wires 400 or 500-512) comprises the following weight percentage: 0.0279 carbon, 0.0002 chromium; 55.93 nickel, 0.0006 copper, 0.0013 cobalt, 0.0062 iron, <0.005 hydrogen, 0.0196 oxygen, balance titanium. Applicant incorporates by reference the American Society for Testing and Materials (ASTM) F2063, Standard Specification for Wrought Nickel-Titanium Shape Memory Alloys for Medical Devices and Surgical Implants.

Such titanium-nickel alloy comprises properties that are favorable for use with anchor implantation in spinal surgical procedures, including biocompatibility, elastic deployment, constancy of stress, physiological compatibility, thermal deployment, dynamic interference, fatigue resistance, hysteresis, Magnetic Resonance Imaging compatibility, high corrosion resistance, and kink resistance. In certain embodiments, Applicant's titanium-nickel alloy is referred to as Nitonol. Table 1 recites exemplary approximate values for certain properties of an elastic alloy wire made of such a titanium-nickel alloy.

TABLE 1
Density: about 6.45 gms/cc
Melting Temperature: about 1240-1310° C.
Resistivity (hi-temp state): about 82 microohm-cm
Resistivity (lo-temp state): about 76 microohm-cm
Thermal Conductivity: about 0.1 W/cm-° C.
Heat Capacity: about 0.077 cal/gm-° C.
Latent Heat: about 5.78 cal/gm, 24.2 J/gm
Magnetic Susceptibility (hi-temp): about 3.8 microemu/gm
Magnetic Susceptibility (lo-temp): about 2.5 microemu/gm
Ultimate Tensile Strength: about 750-1400 MPa or about 110-200 Kpsi
Typical Elongation to Fracture: about 5-10 percent
Typical Yield Strength (hi-temp, above transformation temperature):
about 560 MPa, 80 kpsi
Typical Yield Strength (lo-temp, below transformation temperature):
about 100 MPa, 15 kpsi
Approximate Elastic Modulus (hi-tem): about 75 GPa, 11 Mpsi
Aproximate Elastic Modulus (lo-temp): about 28 GPa, 4 Mpsi
Approximate Poisson's Ratio: about 0.3
Energy Conversion Efficiency: about 5%
Work Output: about 1 Joule/gram
Available Transformation Temperatures: about −100 to +100° C.

With respect to kink resistance, such a titanium-nickel elastic alloy wire has an elastic response to an applied stress such that about all of the strain is recovered upon unloading. To illustrate, the titanium-nickel elastic alloy wire has a springback or elasticity realization of above 5%, such as about an 11%, as compared with 0.5% in the most commonly used medical material, stainless steel. The titanium-nickel elastic alloy wire has a stress-strain inflection point (e.g., point on at which the curvature changes sign) that indicates the presence of an unloading plateau, or a strain range with approximately constant stress.

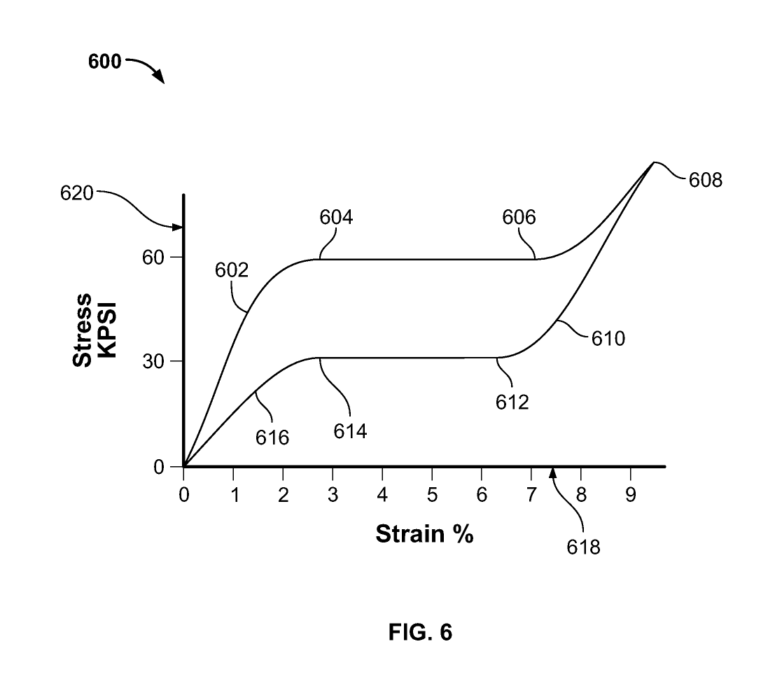

Referring to FIG. 6, a graph 600 illustrates the stress-strain hysteresis curve of such an exemplary titanium-nickel alloy. In FIG. 6, the x-axis 618 is strain in % elongation and the y-axis 620 is stress in units of KPsi. The curve between the origin “0” and point 602 illustrates elastic deformation of the kink resistant titanium-nickel alloy; however, between point 602 to point 604, the strain is no longer proportional to the applied stress. After point 604 the strain remains relatively constant across a large plateau region of strain or deformation until point 606. This plateau region is the loading stress of the material. After point 606, the material begins to deform and beyond point 608, the deformation becomes permanent. In turn, when the applied stress is removed, if no permanent deformation occurred, the materials begins to recover its original shape, following the curve from point 608 to point 610 and then to point 612. The plateau region between point 612 and 614, also known as the unloaded stress region, has a relatively constant stress over a range of strain or deformation. Finally, the curve between 614 to 616 and the origin represents the elastic recovery of the material to its original shape. Here, the loading stress region has a greater magnitude than the unloaded stress region. To illustrate, when the thickness of an exemplary titanium-nickel elastic alloy wire is about 1.62 mm thick, the titanium-nickel elastic alloy wire has the following approximate mechanical properties: 288 Kg break load, 1379 MPa tensile strength, 19.5% elongation, 127 mm gage length, 2.54× Head Spd Pri (mm/min), 102 Kg yield load, yield strength above 400 MPa (e.g., 487 MPa yield strength), and between 35% to 45% cold work such as 39.1% cold work; and the following chemical properties at 23 degrees Celsius: 0.32% permanent set; 8% loading strain; 102 Kg upper plateau load; 487 MPa upper plateau stress; 31.6 Kg lower plateau load; 151 MPa lower plateau stress (restoring strength); upper plateau measured at 4% strain; and lower plateau measured at 4% strain.

In certain embodiments, the elastic alloy wire comprises one or more biocompatible corrosion resistant, and/or antibacterial materials. For example, in certain embodiments, the elastic alloy wire is made of sterilized titanium-nickel alloy and is coated with a germicide such as an anti-biotic-poly-DL-lactic acid (PDLLA) or PDLLA and PDLLA 10% gentamicine coating. In certain embodiments, the titanium-nickel alloy has a surface finish such as an etched and/or other mechanically polished finish or an oxide or pickled finish.

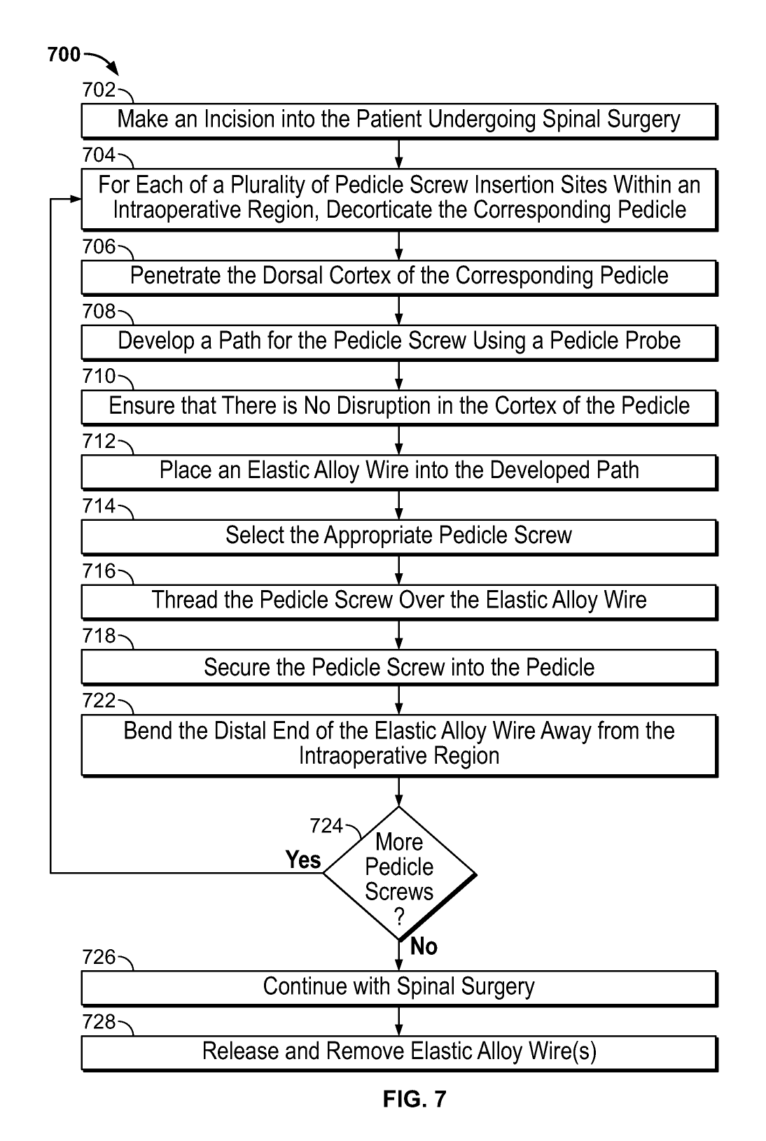

Referring to FIG. 7, a flow chart illustrates an exemplary method for spinal surgery using one or more elastic alloy wires. At step 702, an incision is made into a patient undergoing spinal surgery, such as in the posterior lumbar region of the patient, and tissue is dissected to expose the entry points for the pedicle screw and to provide the required lateral to medial orientation for optimal screw trajectory. In spinal fusion, supplementary bone tissue, is either grafted from the pelvis of the patient (autograft) or previously obtained from a donor (allograft), for use in conjunction with the body's natural bone growth (osteoblastic) processes to fuse the vertebrae.

At step 704, for each of a plurality of pedicle screw insertions within an intraoperative region, a pedicle screw insertion site is decorticated with a burr and high-speed drill or a rongeur. At step 706, a burr or awl is used to penetrate the dorsal cortex of the pedicle. At step 708, a curved or straight pedicle probe is used to develop a path for the pedicle screw through the cancellous bone of the pedicle into the vertebral body. At step 710, after cannulation, a pedicle sounding probe is placed into the pedicle that is then palpated from within to determine whether there is a medial, lateral, rostral or caudal disruption in the cortex of the pedicles. The pedicle sounding probe is also used to verifying that penetration of the ventral cortex of the vertebral body has not occurred. At step 712, Applicant's elastic alloy wire is temporarily placed into the developed path in the pedicle to confirm the trajectory and entry site of the corresponding pedicle screw. For example, the proximal end of the elastic alloy wire is placed into the pilot hole to guide an entry site for the pedicle screw. In certain embodiments, a trocar needle, such as a Jamshidi™ needle creates the pilot hole or is inserted into the pilot hole to help place the elastic alloy wire. Optionally, the elastic alloy wire is screwed into the vertebra, such as by twisting the elastic alloy wire about its long axis (e.g., 412 in FIG. 4A) to push at least a portion of the threading (e.g., 410 in FIG. 4B) into the bony structure of the vertebra and temporarily embed at least a portion of the proximal 402 end of the elastic alloy wire into the bone. Optionally, an X-ray image or other image of the placed elastic alloy wire is taken to compare against desired placement of a corresponding pedicle screw.

Referring to FIGS. 4A and 7, at step 714, the pedicle screw with the appropriate diameter and length is selected. The appropriate diameter is the pedicle screw having a diameter that fits in the developed path but does not fracture the pedicle. The appropriate length of the screw is estimated to produce a depth of about 50-80% of the vertebral body. For example, the appropriate diameter and length for a pedicle screws in the lumbar spine is about 4.5 mm to about 7 mm diameter and a about 35 mm to about 50 mm length. At step 716, the pedicle screw is threaded over the elastic alloy wire. For example, the distal end 406 of the elastic alloy wire is inserted into the lumen of the pedicle screw and the pedicle screw is moved along the length of the elastic alloy wire 400 toward its proximal end 402 until the pedicle screw touches the bone of the vertebra of the patient. At step 718, the pedicle screw is secured into the vertebra (e.g., via top-loading or side-loading). In certain embodiments, the pedicle screw is substantially permanently affixed to a vertebra of the patient.

At step 722, the distal end 406 of the elastic alloy wire 400 is bent away from the intraoperative region (e.g., intraoperative region 524 in FIG. 5B), such as by manually bending the elastic alloy wire away from the intraoperative region and clamping at least a portion of the elastic alloy wire to a surgical drape. In certain embodiments, the elastic alloy wire is bent such that the strain is below the deformation point of the material (e.g., point 608 of FIG. 6). At step 724, a determination is made if more pedicle screws are to be inserted. If more pedicle screws are to be inserted, step 724 proceeds to step 704 and steps 704 to 724 are repeated. If no more pedicle screws are to be inserted, step 724 proceeds to step 726 where the spinal surgery is continued. For example, in spinal fusion, after the pedicle screw placement, the transverse process and the lateral aspects of the facet joints are decorticated, the pedicle screws are connected to a longitudinal construct, usually a rod or a plate, which may be bent to conform to the proper curvature of the spine. The bone graft is then placed. At step 728, the elastic alloy wire(s) is/are removed from the pedicle of the vertebra. For example, if the proximal end of the elastic alloy wire(s) include threading, the elastic alloy wire(s) are untwisted along their long axis to free them from their respective pedicles. Typically, the removed elastic alloy wire(s) return to about their original form without any substantial deformation. In certain embodiments, the elastic alloy wire(s) are reusable and in other embodiments, they are disposable.

The schematic flow chart diagrams included are generally set forth as a logical flow-chart diagram (e.g., FIG. 7). As such, the depicted order and labeled steps are indicative of one embodiment of the presented method. In certain embodiments, other steps and methods are conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types are employed in the flow-chart diagrams, they are understood not to limit the scope of the corresponding method (e.g., FIG. 7). Indeed, some arrows or other connectors may be used to indicate only the logical flow of the method. For instance, an arrow indicates a waiting or monitoring period of unspecified duration between enumerated steps of the depicted method. Additionally, the order in which a particular method occurs may or may not strictly adhere to the order of the corresponding steps shown.

In certain embodiments, individual steps recited in FIG. 7 are combined, eliminated, or reordered. To illustrate, in certain embodiments, after the elastic alloy wire is placed and temporarily secured into the pedicle (step 712) a determination is made whether more pedicle screws will be implanted into the patient (step 724). If yes, the elastic alloy wire is bent away from the intraoperative region (step 722) and another pedicle is decordicated and a respective path is developed for a corresponding pedicle screw (step 704-708). After subsequent elastic alloy wires are secured into respective pedicles (step 712), the elastic alloy wires are optionally released to unbend them. One or more X-ray images are taken to ensure proper placement of each of the elastic alloy wires and spacing between the elastic alloy wires that foretell the location of the pedicle screws. If previously released, the distal ends of one or more elastic alloy wires are bent away from the intraoperative (step 722). For each of the elastic alloy wires, the respective pedicle screws is threaded over the corresponding elastic alloy wires (step 716) and secured into the corresponding pedicles (step 718). The spinal surgery is continued (step 726) and the elastic alloy wires are released and removed (step 728). When spinal surgery is completed, the patient is sutured closed.

While various embodiments have been described above, it should be understood that they have been presented by way of example only, not limitation, and various changes in form and details may be made. Any portion of the apparatus, systems, and/or methods, for example, described herein may be combined in any combination, except mutually exclusive combinations. The embodiments described herein can include various combinations and/or sub-combinations of the functions, components and/or features of the different embodiments described.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

I claim:

1. A method for spinal surgery involving a plurality of pedicle screw insertions, the method comprising, for each of a plurality of pedicle insertion sites within an intraoperative region:
temporarily placing a proximal end of an elastic alloy wire into a respective pedicle of a vertebra, wherein the elastic alloy wire:
has a distal end opposite to the proximal end; and
does not kink when bent;
inserting the distal end of the elastic alloy wire into a lumen of a respective pedicle screw;
moving the respective pedicle screw along the elastic alloy wire toward the proximal end of the elastic alloy wire; and
removing the elastic alloy wire from the pedicle of the vertebra.

2. The method of claim 1, wherein the elastic alloy wire is a titanium-nickel alloy wire.

3. The method of claim 2, wherein the titanium-nickel alloy wire comprises between 54 to 57 weight percent nickel.

4. The method of claim 1, further comprising, prior to placing the proximal end of the elastic alloy wire into the pedicle, using a pedicle probe to develop a pilot hole for the elastic alloy wire.

5. The method of claim 1, further comprising, prior to inserting the distal end of the elastic alloy wire into the lumen, twisting the elastic alloy wire along its long axis to push at least a portion of a threading at the proximal end of the elastic alloy wire into the pedicle.

6. The method of claim 1, further comprising, prior to removing the elastic alloy wire, using an imaging device to determine at least one of:
a location of the proximal end of the elastic alloy wire placed into the respective pedicle;
a trajectory along the length of the elastic alloy wire placed into the respective pedicle; and
a spacing between the plurality of the elastic alloy wires placed into the respective pedicles.

7. The method of claim 1, further comprising bending one or more distal ends of respective elastic alloy wires away from the intraoperative region.

8. A method for spinal surgery involving inserting a plurality of anchors into a plurality of respective pedicles, the method comprising, for each of the plurality of anchors:
temporarily placing a proximal end of a titanium-nickel alloy wire into a respective pedicle of a vertebra, wherein:
the proximal end includes threading;
the titanium-nickel alloy wire has a distal end opposite to the proximal end;
and
the titanium-nickel alloy wire does not kink when bent;
twisting the titanium-nickel alloy wire along its long axis to push at least a portion of the threading into the pedicle;
inserting the distal end of titanium-nickel alloy wire into a lumen of a respective anchor;
moving the respective anchor along the titanium-nickel alloy wire toward the proximal end; and
untwisting the titanium-nickel alloy wire free from the pedicle of the vertebra.

9. The method of claim 8, further comprising, prior to removing the titanium-nickel alloy wire, using an imaging device to determine at least one of:
a location of the proximal end of the titanium-nickel alloy wire placed into the respective pedicle;
a trajectory along the titanium-nickel alloy wire placed into the respective pedicle;
and
a spacing between the plurality of the titanium-nickel alloy wires placed into the respective pedicles.

10. The method of claim 8, further comprising bending one or more distal ends of respective titanium-nickel alloy wires away from an intraoperative region during the spinal surgery.

11. The method of claim 8, wherein the titanium-nickel alloy wire comprises between 54 to 57 weight percent nickel.

12. The method of claim 8, wherein the titanium-nickel alloy wire has an elasticity realization of above five percent, enabling the distal end of the titanium-nickel alloy wire to be bent away from an intraoperative region during the spinal surgery without forming a kink anywhere along a length of the titanium-nickel alloy wire.

13. A wire to guide an entry site of a pedicle screw used in spinal surgery, the wire comprising:
an elastic alloy having an elasticity that enables a distal end of the wire to be bent away from an intraoperative region during the spinal surgery without forming a kink along a length of the wire;
threading at a proximal end of the wire, wherein the wire is dimensioned to:
fit into a lumen of a pedicle screw; and
allow the pedicle screw to glide along the length of the wire from a distal end toward the proximal end of the wire.

14. The wire of claim 13, wherein the wire has an elasticity realization of above five percent.

15. The wire of claim 13, wherein the wire is made of a titanium-nickel alloy comprising between 54 to 57 weight percent nickel.

16. The wire of claim 13, wherein a diameter of the wire lies in a range of 1 mm to 3 mm.

17. The wire of claim 13, wherein a tip of the wire at the proximal end is selected from the group consisting of:
a sharp tip;
a beveled tip;
a blunt tip;
a rounded tip; and
a combination thereof.

18. The wire of claim 13, wherein a length of the wire lies in a range of 400 mm to 700 mm.

19. The wire of claim 13, further comprising a surface coating that is at least: biocompatible and germicidal.

20. The wire of claim 13, wherein the wire has a yield strength above 400 MPa.
